(19) United States
(12) Patent Application Publication
(10) Pub. No.: US 2018/0168986 A1
(43) Pub. Date: Jun. 21, 2018

(54) AGENT AND METHOD FOR HAIR CLEANSING

(71) Applicant: Henkel AG & Co. KGaA, Duesseldorf (DE)

(72) Inventors: Thorsten Knappe, Schenefeld (DE); Pamela Kaftan, Hamburg (DE); Ulrike Heinsohn, Hamburg (DE)

(73) Assignee: Henkel AG & Co. KGaA, Duesseldorf (DE)

(21) Appl. No.: 15/796,884

(22) Filed: Oct. 30, 2017

(30) Foreign Application Priority Data
Dec. 20, 2016 (DE) 10 2016 225 660.2

(51) Int. Cl. A61K 8/73; A61K 8/34; A61K 8/04; A61K 8/02; A61Q 5/02; A61K 8/89; A61K 8/41

(52) U.S. Cl. CPC A61K 8/732; A61K 8/34; A61K 8/046; A61K 2800/43; A61Q 5/02; A61K 8/89; A61K 8/416; A61K 8/0241

(57) ABSTRACT

A cosmetic composition is provided herein. The cosmetic composition includes, relative to the total weight of the cosmetic composition, a) from about 2.0 to about 10 wt. % starch particles. The starch particles include, relative to the weight of the starch particles, a1) from about 65 to about 98 wt. % starch, and a2) from about 1.0 to about 30 wt. % pigment. The cosmetic composition further includes, relative to the total weight of the cosmetic composition, b) from about 2.0 to about 10 wt. % ethanol. The cosmetic composition further includes, relative to the total weight of the cosmetic composition, c) from about 80 to about 96 wt. % propellant.

### CROSS-REFERENCE TO RELATED APPLICATION

This application claims priority to German Patent Application No. 10 2016 225 660.2, filed Dec. 20, 2016, which is incorporated herein by reference in its entirety.

### TECHNICAL FIELD

The application relates to agents and methods for cleansing keratinous fibers, more particularly human hair.

### BACKGROUND

Keratinous fibers are usually cleansed by employing the combined effect of water, soap and mechanical work. For example, hair washing involves first applying a tenside-containing shampoo to damp hair and massaging the shampoo into the hair. If the hair is then rinsed with water, the impurities detached by employing the water, the shampoo and/or the mechanical effect are rinsed out of the hair, the hair thus being cleansed in this manner.

If there is no water available for hair cleansing or the hair cannot be cleansed in the conventional manner for lack of time, hair cleansing by employing a dry shampoo is suitable as an alternative to water-based hair cleansing.

Dry shampoos contain, as an essential active ingredient, a particulate carrier material which, due to its adsorption properties, is able to bind impurities such as fats or sebum present on the hair or scalp. Modified starches are primarily used as carrier materials. When the hair is combed or brushed after the dry shampoo has been applied, the adsorbed impurities are removed from the hair along with the powdery carrier material.

The aerosol spray is a preferred packaging form for dry shampoo. With similar products, the powder suspended in a liquid phase is sprayed onto the hair by employing a propellant. Such agents are described, for example, in international patent applications WO 2013/143792 A2 and WO 2013/143935 A2.

Dry shampoo not completely removed from the hair can cause a bloom on the hair or adhere to the hair in the form of visible particles. Both these effects are among the essential disadvantages of using dry shampoo. According to international patent application WO 2011/056625 A2, this problem can be solved by using a combination of starch and clay as the particulate carrier material. This technical solution has proven to be less than ideal. The problem of bloom formation cannot be completely resolved through this technical approach. Likewise, using a mixture of particulate carrier materials increases the complexity and costs of the cosmetic product and leads, particularly in the aerosol spray packaging form, to difficulties with application.

### BRIEF SUMMARY

A cosmetic composition is provided herein. The cosmetic composition includes, relative to the total weight of the cosmetic composition, a) from about 2.0 to about 10 wt. % starch particles. The starch particles include, relative to the weight of the starch particles, a1) from about 65 to about 98 wt. % starch, and a2) from about 1.0 to about 30 wt. % pigment. The cosmetic composition further includes, relative to the total weight of the cosmetic composition, b) from about 2.0 to about 10 wt. % ethanol. The cosmetic composition further includes, relative to the total weight of the cosmetic composition, c) from about 80 to about 96 wt. % propellant.

The problem addressed by the present disclosure was therefore that of providing a dry shampoo that has a high adsorption capacity and good application characteristics when applied in the form of an aerosol spray which, when applied to a lesser extent, tends to form visible residues or blooms on the hair. The problem was solved by employing using specific starch particles.

The present application provides:

1. A cosmetic composition containing, relative to its total weight

a) from about 2.0 to about 10 wt. % starch particles comprising, relative to the weight of the starch particles
a1) from about 65 to about 98 wt. % starch
a2) from about 1.0 to about 30 wt. % pigment

b) from about 2.0 to about 10 wt. % ethanol

c) from about 80 to about 96 wt. % propellant.

2. Cosmetic composition according to Item 1, wherein the weight proportion of the starch particles relative to the total weight of the cosmetic composition is from about 3.0 to about 8.0 wt. % and more particularly from about 4.0 to about 5.0 wt. %.

3. Cosmetic composition according to one of the preceding items, wherein the starch particles comprise, relative to the total weight thereof, from about 70 to about 96 wt. % of starch, preferably from about 80 to about 94 wt. % of starch.

4. Cosmetic composition according to one of the preceding items, wherein the starch particles contain starch in the form of rice starch.

5. Cosmetic composition according to one of the preceding items, wherein the starch particles comprise, relative to the total weight thereof, from about 1.5 to about 25 wt. % of pigment, preferably from about 2.0 to about 18 wt. % of pigment.

6. Cosmetic composition according to one of the preceding items, wherein the starch particles comprise, as a pigment, a synthetic inorganic pigment, preferably a synthetic inorganic pigment from the group of metal oxides, and more particularly a synthetic inorganic pigment from the group of iron oxides.

7. Cosmetic composition according to one of the preceding items, wherein the starch particles also comprise, relative to the total weight thereof
a3) from about 0.5 to about 5.0 wt. % at 20° C. and 1,013 hPa liquid oil.

8. Cosmetic composition according to Item 7, wherein the starch particles, relative to the total weight thereof, comprise from about 1.0 to about 4.0 wt. %, preferably from about 1.5 to about 3.0 wt. % at 20° C. and 1,013 hPa liquid oil.

9. Cosmetic composition according to one of Items 7 or 8, wherein the starch particles comprise, an oil which is liquid at 20° C. and 1,013 hPa, a silicone oil, preferably from the group of dialkyl and alkylaryl siloxanes, more preferably from the group of polydimethyl siloxane, cyclopenta siloxane, cyclohexa siloxane and methylphenyl polysiloxane, and most preferably from the group of polydimethylsiloxanes.

10. Cosmetic composition according to one of the preceding items, wherein the starch particles also comprise, relative to the total weight thereof
a4) from about 0.01 to about 1.0 wt. % cationic tenside.

11. Cosmetic composition according to Item 10, wherein the starch particles comprise, relative to the total weight thereof, from about 0.02 to about 0.7 wt. % cationic tenside, preferably from about 0.05 to about 0.5 wt. % cationic tenside.

12. Cosmetic composition according to one of Items 10 or 11, wherein the starch particles comprise, as a cationic tenside, a quaternary ammonium compound, preferably a cationic tenside from the group of cetyltrimethyl ammonium chloride, stearyltrimethyl ammonium chloride, distearyldimethyl ammonium chloride, lauryl dimethyl ammonium chloride, lauryldimethyl benzyl ammonium chloride and tricetylmethyl ammonium chloride, and most preferably cetyltrimethyl ammonium chloride.

13. Cosmetic composition according to one of the preceding items, wherein the starch particles have a coating of pigment.

14. Cosmetic composition according to one of the preceding items, wherein the starch particles are produced by coating starch with pigment.

15. Cosmetic composition according to one of the preceding items, wherein the composition comprises, relative to the total weight thereof, from about 3.0 to about 8.0 wt. % and preferably from about 4.0 to about 6.0 wt. % ethanol.

16. Cosmetic composition according to one of the preceding items, wherein the composition comprises, relative to the total weight thereof, from about 82 to about 94 wt. % of propellant, preferably from about 86 to about 92 wt. % of propellant.

17. Cosmetic composition according to one of the preceding items, wherein the composition comprises a propellant from the group of propane, propane/butane mixtures and dimethyl ether, more particularly from the group of propane/butane mixtures.

18. Use of a cosmetic composition according to one of Items 1 to 17 for cleansing keratinous fibers, more particularly human hair.

19. Method for the temporary shaping of keratinous fibers, more particularly human hair, wherein the cosmetic composition according to one of Items 1 to 18 is applied to keratinous fibers.

20. Method according to Item 19, wherein the cosmetic composition is applied to the hair, left for a period of from about 1 to about 10 minutes and then removed from the hair.

21. Method for cleansing keratinous fibers, more particularly human hair, comprising the following steps:

i) Provision of two or more cosmetic compositions, comprising

a) from about 2.0 to about 10 wt. % starch particles comprising, relative to the weight of the starch particles
a1) from about 65 to about 98 wt. % starch
a2) from about 1.0 to about 30 wt. % pigment

b) from about 2.0 to about 10 wt. % ethanol

c) from about 80 to about 96 wt. % propellant,

wherein the two or more cosmetic compositions differ in terms of
the weight proportion of the pigment on the starch particle and/or
the chemical composition of the pigment and/or
the physical structure of the pigment

ii) application of the cosmetic composition on the keratinous fibers;

iii) in some cases at least the partial removal of the cosmetic composition from the keratinous fibers.

The agents as contemplated herein exist preferably in the form of a dispersion of starch particles in a liquid phase from ethanol and propellant.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

A first essential constituent of compositions as contemplated herein is the starch particle, which in turn comprises pigment in addition to starch. “Particles” are solids which exist as particulate at 25° C. and 1013 mbar. As contemplated herein, the starch particles preferably a mean particle diameter of from about 5 to about 100 μm, and more preferably from about 10 to about 75 μm.

For the applicability and cosmetic effect of the cosmetic composition, it has proven advantageous for the weight proportion of the starch particles, relative to the total weight of the cosmetic composition, to be limited to values from about 3.0 to about 8.0 wt. % and more particularly from about 4.0 to about 5.0 wt. %. Corresponding compositions are preferred.

In addition to the weight proportion of the starch particles relative to the total weight of the cosmetic composition, the composition of the starch particles per se has proven relevant to the cosmetic effect.

The starch particles comprise, as their two essential constituents, starch and pigment.

Starch is a reserve carbohydrate, which is stored by many plants in the form of, usually, from about 1 to about 200 μm starch grains (granulate) in various plant parts, e.g. bulbs or roots, grain seeds, fruits, and also in the core. Starch belongs to the family of homoglycans and a poly-condensation product of D-glucose. Starch of three structurally different polymers of d-glucopyranose, specifically amylose, amylopectin and a so-called intermediate fraction.

A starch compound usable as contemplated herein is selected from at least one—possibly modified—poly-condensation product of D-glucose, obtained from the starch of potatoes, corn, rice, peas, acorns, chestnuts, barley, wheat, bananas, sago, sorghum, oats, rye, beans, batata, maranta or manioc. More preferably, the agent as contemplated herein contains at least one starch compound, which his tapioca starch, potato starch, corn starch or rice starch. As contemplated herein, the agent likewise comprises mixtures of the aforementioned starch compounds. Most preferably, the starch compound is rice starch.

Particularly advantageous cosmetic compositions contain starch particles, which in turn contain, relative to the total weight thereof, from about 70 to about 96 wt. % of starch, more particularly from about 80 to about 94 wt. % of starch, most preferably rice starch. The starch particles contained in the cosmetic compositions contain pigment in addition to the starch. With respect to the cleaning effect of the cosmetic composition and the optical cosmetic effect thereof, it has proven advantageous for the starch particles to contain, relative to the total weight thereof, from about 1.5 to about 25 wt. % of pigment, preferably from about 2.0 to about 18 wt. % of pigment. Corresponding starch particles are therefore preferred.

The used pigment is preferably insoluble in water. In an embodiment as contemplated herein, the pigment is selected from the group formed from mineral pigments, metal pigments and pearl luster pigments.

The use of inorganic pigments is preferred. Said pigments comprise naturally occurring pigments and those obtainable through mechanical treatment, as well as synthetic pigments.

Mineral pigments or natural inorganic pigments consist mainly of sulfides and oxides. Preferred natural pigments are Ocker (Fe(OOH); Pigment Yellow 43), burned Siena (Fe₂O₃; Pigment Red 102), Umbra (Fe₂O₃.xMnO₂; Pigment Brown 7:x), cinnabar (β-HgS, PR 106), lapis lazuli (Ultramarine, Na₆Al₆Si₆O₂₄.Na₂Sₙ; Pigment Blue 29), Azurit (basic copper carbonate, Cu₃[OH/CO₃]₂; PB 30), green earth (FeO-containing silicate; Pigment Green 23), malachite (Cu₂[(OH)₂, CO₃]) and carbon black (carbon (graphite), Pigment Black 9).

Powdery metals or metal alloys are referred to as metal pigments or metal effect pigments. As contemplated herein, aluminum pigments (aluminum bronze), as well as brass pigments (so-called gold bronzes), i.e. copper/aluminum alloys or copper/zinc alloys, wherein the term “bronze” should not be considered in the narrow chemical sense in this context, are particularly advantages. Typical metal pigments are

Aluminum bronzes (Al; silver color);

Gold bronzes (Cu, Cu—Al- or Cu—Zn-alloy; natural color shades)

(Cu; copper color)

(90% Cu-10% Zn; bleach gold)

(85% Cu-15% Zn; rich copper gold)

(70% Cu-30% Zn; copper gold)

Silver bronze (Cu—Zn—Ni; silver)

Fire-dyed bronzes (oxidized Cu—Zn; temper colors, such as lemon, fire red, sea green)

Patent bronzes (Cu—Zn—(Ni)+dyes; various colors)

Pearlescent pigments are gloss pigments, which consist of multiple layers of different refractive index. Pearlescent pigments preferred as contemplated herein are magnesium stearate, zinc stearate and lithium stearate or ethylene glycoldistearate and/or polyethylene terephthalate, as well as pearlescent pigments comprising essentially glimmer, titanium dioxide (titanium dioxide glimmer), bismut oxychloride or guanine, and which can also be covered with colored oxide layers (e.g. iron oxide or chromium oxide) .

Glimmer-based and glimmer/metal oxide-based pearlescent pigments are particularly preferred pearlescent pigments as contemplated herein. Glimmers are layer silicates. The most important representatives of such silicates as muscovite, phlogopite, paragonite, biotite, lepidolithe and margarite. To produce the pearlescent pigments in conjunction with metal oxides, the glimmer, primarily muscovite or phlogopite, is coated with a metal oxide. Suitable metal oxides include TiO₂, Cr₂O₃ and Fe₂O₃. Interference pigments and pearlescent pigments are obtained, as pearlescent pigments preferred as contemplated herein, by coating. Said pearlescent pigment types have color effects, in addition to a glittering optical effect. Moreover, the pearlescent pigments usable as contemplated herein can additionally contain a color pigment, which is not derived from a metal oxide.

Most preferred pearlescent pigments are pigments which are marketed by Merck under the trade name Colorona®, wherein the Colorona® pigments contain red-brown (from about 47 to about 57 wt. % Muscovit Mica (KH₂(AlSiO₄)₃), from about 43 to about 50 wt. % Fe₂O₃ (INCI: Iron Oxides CI 77491), <3 wt. % TiO₂ (INCI: Titanium Dioxide CI 77891), Colorona® Blackstar Blue (from about 39 to about 47 wt. % Muscovit Mica (KH₂(AlSiO₄)₃), from about 53 to about 61 wt. % Fe₃O₄ (INCI: Iron Oxides CI 77499)), Colorona® Siena Fine from about (from about 35 to about 45 wt. % Muscovit Mica (KH₂(AlSiO₄)₃), from about 55 to about 65 wt. % Fe₂O₃ (INCI: Iron Oxides CI 77491)), Colorona® Aborigine Amber (from about 50 to about 62 wt. % Muscovit Mica (KH₂(AlSiO₄)₃), from about 36 to about 44 wt. % Fe₃O₄ (INCI: Iron Oxides CI 77499), from about 2 to about 6 wt. % TiO₂ (INCI: Titanium Dioxide CI 77891)), Colorona® Patagonian Purple (from about 42 to about 54 wt. % Muscovit Mica (KH₂(AlSiO₄)₃), from about 26 to about 32 wt. % Fe₂O₃ (INCI: Iron Oxides CI 77491), from about 18 to about 22 wt. % TiO₂ (INCI: Titanium Dioxide CI 77891), from about 2 to about 4 wt. % Prussian blue (INCI: Ferric Ferrocyanide CI 77510)), Colorona® Chameleon (from about 40 to about 50 wt. % Muscovit Mica (KH₂(AlSiO₄)₃), from about 50 to about 60 wt. % Fe₂O₃ (INCI: Iron Oxides CI 77491)) and Silk® Mica (>98 wt. % Muscovit Mica (KH₂(AlSiO₄)₃)).

Synthetic inorganic pigments are obtained in particular by chemical and/or physical conversion (digestion, precipitation, annealing). Including in particular—white pigments (titanium dioxide (TiO₂), Pigment White PW 6; zinc sulfide (ZnS), PW 7; zinc oxide (ZnO), PW 4; antimony white (Sb₂O₃), PW 11; lithopones (ZnS/BaSO₄), PW 5; beach white (2PbCO₃.Pb(OH)₂), PW 1),
subordinately, white fillers (calcium carbonate, PW 18; talcum, PW 26 and barium sulfate, PW 21);
black pigments (manganese black, spinel black and pigment soots (graphite hydrocarbon);
gloss pigments (absorption pigments, metal pigments or metal effect pigments and pearlescent pigments), as well as
inorganic color pigments (iron oxide pigments, iron blue pigments, ultramarine pigments, as well as the bleach chromate pigments, chromium oxide pigments, cadmium pigments and bismutvanadate pigments, which are less suitable due to their toxicological properties).

A group of particularly preferred pigments is formed from the chromophoric synthetic iron oxides. Particularly preferred representatives from this substance class are Pigment Brown 6 (CI No 77491), Pigment Red 101 (CI No 77491), Pigment Yellow 42 (CI No 77492), Pigment Black 11 (CI No 77499), as well as mixtures of said pigments.

Most preferred cosmetic compositions contain starch particles, which contain in turn, as a pigment, a synthetic inorganic pigment, preferably a synthetic inorganic pigment from the group of metal oxides, and more particularly a synthetic inorganic pigment from the group of iron oxides.

The starch particles contained in the cosmetic agents as contemplated herein preferably have a core made of starch and a pigment coating, which surrounds said core at least partially. The starch particles are preferably produced by coating starch with the pigment. In summary, a particularly preferred cosmetic composition contains, relative to the total weight thereof

a) from about 3.0 to about 8.0 wt. % starch particles comprising, relative to the weight of the starch particles

a1) from about 70 to about 96 wt. % starch

a2) from about 1.5 to about 25 wt. % synthetic inorganic product from the group of iron oxides

b) from about 3.0 to about 8.0 wt. % ethanol

c) from about 82 to about 94 wt. % propellant.

In addition to the starch and the pigment, preferred starch particles contain further additional active ingredients and excipients.

A first group of preferred active ingredients and excipients is the oils that are liquid at 20° C. and 1,013 hPa. Said oils act primarily as release agents and prevent the starch particles from clumping in the cosmetic composition:

The weight proportion of the oil, which is liquid at 20° C. and 1,013 hPa, relative to the total weight of the starch particles is preferably from about 0.5 to about 5.0 wt. %, more preferably from about 1.0 to about 4.0 wt. % and most preferably from v1.5 to about 3.0 wt. %.

According to a preferred embodiment, the cosmetic oil, which is liquid at 20° C. and 1,013 hPa, is selected from the group of

(i) volatile cyclic silicone oils, more particularly cyclotrisiloxane, cyclotetrasiloxane, cyclopentasiloxane and cyclohexasiloxane, and linear silicone oils having 2 to 10 siloxane units, more particularly hexamethyl disiloxane, octamethyltrisiloxane, decamethyl tetrasiloxane;

(ii) volatile non-silicone oils, more particularly liquid paraffin oils and iso-paraffin oils, such as isodecane, isoundecane, isododecane, isotridecane, isotetradecane, isopentadecane, isohexadecane and isoeicosane;

(iii) non-volatile silicone oils, more particularly higher-molecular linear polyalkyl siloxanes;

(iv) non-volatile non-silicone oils, more particularly the esters of linear or branched saturated or unsaturated C₂₋₃₀ fatty alcohols with linear or branched saturated or unsaturated C₂₋₃₀ fatty acids, which can be hydroxylated, the C₈-C₂₂ fatty alcohol esters of monovalent or polyvalent C₂-C₇ hydroxycarbonic acids, the triethylcitrates, the branched saturated or unsaturated C₆₋₃₀ fatty alcohols, the mono-, di- and triglycerides of linear or branched, saturated or unsaturated, likewise hydroxylated C₈₋₃₀ fatty acids, the dicarbonic acid esters of linear or branched C2-C₁₀alkanols, the addition products of ethylene oxide and/or propylene oxide to monovalent or polyvalent C3.22 alkanols, which may be esterified, the symmetrical, asymmetrical or cyclic esters of carbonic acids with fatty alcohols, the esters of dimer unsaturated C₁₂₋₂₂-fatty acids with monovalent, linear, branched and cyclic C₂₋₁₈-alkanols or C₂₋₆ alkanols, the benzoic acid esters of linear or branched C₈₋₂₂ alkanols, such as benzoic acid C₁₂₋₁₅ alkyl ester and benzoic acid isoctyldodecyl ester, the synthetic hydrocarbons, such as polyisobutes and polydecenes, the alicyclic hydrocarbons;

(v) the mixtures thereof.

The use of volatile cosmetic oils, more particularly those having a vapor pressure of 0.01 kPa at 20° C., is particularly preferred. From a technical perspective, the use of oils from the group of silicone oils, preferably from the group of dialkyl- and alkylaryl siloxanes is advantageous, more preferably from the group of polydimethylsiloxane, cyclopentasiloxane, cyclohexasiloxane, and methylphenylpolysiloxane, most preferably from the group of polydimethylsiloxanes.

A second group of optional active ingredients and excipients which can be contained in the starch particles is the cationic tensides.

The weight proportion of the cationic tenside relative to the total weight of the starch particles is preferably from about 0.01 to about 1.0 wt. %, more preferably from about 0.02 to about 0.7 wt. % and most preferably from about 0.05 to about 0.5 wt. %.

Preferred cationic tensides are selected from the group of quaternary ammonium compounds. Preferred quaternary ammonium compounds are ammonium compounds and ammonium halogenides, more particularly chlorides and bromides, such as alkyltrimethylammoniumchlorides, dialkyldimethylammoniumchlorides and trialkylmethylammoniumchlorides, e.g. cetyltrimethylammoniumchloride, stearyltrimethylammoniumchloride, distearyldimethylammoniumchloride, lauryldimethylammoniumchlorids, lauryldimethylbenzylammoniumchloride and tricetylmethylammoniumchloride, as well as the imidazolium compounds known under the INCI trade names of Quaternium-27 and Quaternium-83. The use of cetyltrimethyl ammonium chloride is most preferred.

Optional, other suitable active ingredients or excipients which can be contained in the cosmetic components are more particularly nourishing agents.

As the nourishing agent, the agent can contain at least one protein hydrolysate and/or a derivate thereof, for example. Protein hydrolysates are product mixtures obtained through the acidically, basically or enzymatically catalyzed decomposition of proteins. As contemplated herein, the expression protein hydrolysates also includes total hydrolysates, as well as individual amino acids and the derivatives thereof, as well as mixtures of various amino acids. The molecular weight of the protein hydrolysates usable as contemplated herein is between about 75, the molecular weight for glycine, and about 200,000, preferably from about 75 to about 50,000 and most preferably from about 75 to about 20,000 Dalton.

The agent as contemplated herein can also contain, as a nourishing agent a vitamin, a provitamin, a vitamin precursor and/or a derivative thereof. As contemplated herein, such vitamins, provitamins and vitamin precursors are usually assigned to the groups A, B, C, E, F and H.

Other nourishing agents are panthenol, coffein, nicotinamide and sorbitol.

The agent as contemplated herein can also contain, as a nourishing agent, a plant extract, as well as mono- and/or oligosaccharides and/or lipids.

The composition of some other preferred cosmetic compositions can be found in the table below (values in wt. % relative to the total weight of the cosmetic composition and/or relative to the weight of the starch particles).

| | Formula 1 | Formula 2 | Formula 3 | Formula 4 | Formula 5 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |
| | Formula 6 | Formula 7 | Formula 8 | Formula 9 | Formula 10 |
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |
| | Formula 11 | Formula 12 | Formula 13 | Formula 14 | Formula 15 |
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides

| | Formula 16 | Formula 17 | Formula 18 | Formula 19 | Formula 20 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides

| | Formula 21 | Formula 22 | Formula 23 | Formula 24 | Formula 25 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

**oil, which is liquid at 20° C. and 1,013 hPa, preferably silicone oil

| | Formula 26 | Formula 27 | Formula 28 | Formula 29 | Formula 30 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

**oil, which is liquid at 20° C. and 1,013 hPa, preferably silicone oil

| | Formula 31 | Formula 32 | Formula 33 | Formula 34 | Formula 35 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides
**oil, which is liquid at 20° C. and 1,013 hPa, preferably silicone oil

| | Formula 36 | Formula 37 | Formula 38 | Formula 39 | Formula 40 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides
**oil, which is liquid at 20° C. and 1,013 hPa, preferably silicone oil

| | Formula 41 | Formula 42 | Formula 43 | Formula 44 | Formula 45 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |
| | Formula 46 | Formula 47 | Formula 48 | Formula 49 | Formula 50 |
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |
| | Formula 51 | Formula 52 | Formula 53 | Formula 54 | Formula 55 |
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides

| | Formula 56 | Formula 57 | Formula 58 | Formula 59 | Formula 60 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides

| | Formula 61 | Formula 62 | Formula 63 | Formula 64 | Formula 65 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

**oil, which is liquid at 20° C. and 1,013 hPa, preferably silicone oil

| | Formula 66 | Formula 67 | Formula 68 | Formula 69 | Formula 70 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

**oil, which is liquid at 20° C. and 1,013 hPa, preferably silicone oil

| | Formula 71 | Formula 72 | Formula 73 | Formula 74 | Formula 75 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides
**oil, which is liquid at 20° C. and 1,013 hPa, preferably silicone oil

| | Formula 76 | Formula 77 | Formula 78 | Formula 79 | Formula 80 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Cationic | 0.01 to 1.0 | 0.02 to 0.7 | 0.02 to 0.7 | 0.02 to 0.7 | 0.05 to 0.5 |
| tenside | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment, preferably a synthetic inorganic pigment from
the group of metal oxides and, more particularly a synthetic inorganic
pigment from the group of iron oxides
**oil, which is liquid at 20° C. and 1,013 hPa, preferably silicone oil

| | Formula 81 | Formula 82 | Formula 83 | Formula 84 | Formula 85 |
|---|---|---|---|---|---|
| Starch particles, | 2.0 to 10 | 3.0 to 8.0 | 3.0 to 8.0 | 3.0 to 8.0 | 4.0 to 5.0 |
| comprising | | | | | |
| Rice starch | 65 to 98 | 70 to 96 | 70 to 96 | 80 to 94 | 80 to 94 |
| Pigment* | 1.0 to 30 | 1.5 to 25 | 1.5 to 25 | 2.0 to 18 | 2.0 to 18 |
| Oil** | 0.5 to 5.0 | 0.5 to 5.0 | 1.0 to 4.0 | 1.0 to 4.0 | 1.5 to 3.0 |
| Cationic | 0.01 to 1.0 | 0.02 to 0.7 | 0.02 to 0.7 | 0.02 to 0.7 | 0.05 to 0.5 |
| tenside*** | | | | | |
| Ethanol | 2.0 to 10 | 2.0 to 10 | 2.0 to 10 | 3.0 to 8.0 | 4.0 to 6.0 |
| Propellant | 80 to 96 | 82 to 94 | 82 to 94 | 82 to 94 | 86 to 92 |
| Optional additives | ad 100 | ad 100 | ad 100 | ad 100 | ad 100 |

*synthetic inorganic pigment from the group of iron oxides, preferably Pigment Brown 6
(CI No 77491), Pigment Red 101 (CI No 77491), Pigment Yellow 42 (CI No 77492),
Pigment Black 11 (CI No 77499), as well as mixtures of said pigments.
**oil, which is liquid at 20° C. and 1,013 hPa, from the group of dimethicones
***Cetrimonium chloride

As stated at the outset, the cosmetic compositions as contemplated herein are particularly suitable for cleansing keratinous fibers, more particularly human hair. A corresponding use of said agents is a further subject matter of the present disclosure.

To cleanse keratinous fibers, the cosmetic composition is applied to said fibers. In a further step, the composition can subsequently be removed again from the keratinous fiber at least partially. This can be done, for example, through mechanical effect on the fibers, more particularly by combing or brushing the fibers. Alternatively, the cosmetic composition can be blown out of the hair by employing a hairdryer, for example.

If, after application, the cosmetic agent is removed from the keratinous fibers at least partially, the exposure time of the composition on the fibers is preferably between about 1 and about 10 minutes.

As also stated at the outset, an essential advantage of the cosmetic compositions as contemplated herein is cleansing the hair without the formation of visible residues or blooms on the hair following application. This effect is caused by the combination of starch and pigment in the starch particles referred to as the cosmetic composition.

This active ingredient combination offers advantages going beyond the cosmetic effect described above. For example, the used pigment can be matched to the cosmetic requirements of the user concerned due to its optical effect. A particularly preferred cosmetic process therefore comprises the following steps:

iv) Provision of two or more cosmetic compositions, comprising

a) from about 2.0 to about 10 wt. % starch particles comprising, relative to the weight of the starch particles
a1) from about 65 to about 98 wt. % starch
a2) from about 1.0 to about 30 wt. % pigment

b) from about 2.0 to about 10 wt. % ethanol

c) from about 80 to about 96 wt. % propellant,

wherein the two or more cosmetic compositions differ in terms of
the weight proportion of the pigment on the starch particle and/or
the chemical composition of the pigment and/or
the physical structure of the pigment

v) application of the cosmetic composition on the keratinous fibers;

vi) in some cases at least the partial removal of the cosmetic composition from the keratinous fibers.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition comprising, relative to the total weight of the cosmetic composition:
a) from about 2.0 to about 10 wt. % starch particles comprising, relative to the weight of the starch particles;
a1) from about 65 to about 98 wt. % starch, and
a2) from about 1.0 to about 30 wt. % pigment
b) from about 2.0 to about 10 wt. % ethanol; and
c) from about 80 to about 96 wt. % propellant.

2. Cosmetic composition according to claim 1, wherein the weight proportion of the starch particles relative to the total weight of the cosmetic composition is from about 3.0 to about 8.0 wt. %.

3. Cosmetic composition according to claim 1, wherein the starch particles comprise, relative to the total weight thereof, from about 70 to about 96 wt. % of starch.

4. Cosmetic composition according to claim 1, wherein the starch particles comprise, relative to the total weight thereof, from about 1.5 to about 25 wt. % of pigment.

5. Cosmetic composition according to claim 1, wherein the starch particles have a coating of pigment.

6. Cosmetic composition according to claim 1, wherein the starch particles are produced by coating starch with pigment.

7. Cosmetic composition according to claim 1, wherein the composition comprises, relative to the total weight thereof, from about 3.0 to about 8.0 wt. % of ethanol.

8. Cosmetic composition according to claim 1, wherein the composition comprises, relative to the total weight thereof, from about 82 to about 94 wt. % of propellant.

9. Cosmetic composition according to claim 1, wherein the cosmetic composition is utilized for cleansing keratinous fibers.

10. A method for the cleansing of keratinous fibers wherein the method comprises applying the cosmetic composition according to claim 1 to keratinous fibers.

11. Cosmetic composition according to claim 1, wherein the starch particles comprise starch in the form of rice starch.

12. Cosmetic composition according to claim 1, wherein the starch particles comprise, as a pigment, a synthetic inorganic pigment.

13. Cosmetic composition according to claim 1, wherein the starch particles also comprise, relative to the total weight thereof:
a3) from about 0.5 to about 5.0 wt. % of an oil which is liquid at 20° C. and 1,013 hPa.

14. Cosmetic composition according to claim 13, wherein the oil comprises a silicone oil.

15. Cosmetic composition according to claim 14, wherein the silicone oil is selected from the group of polydimethylsiloxanes.

16. Cosmetic composition according to claim 1, wherein the starch particles also comprise, relative to the total weight thereof:
a4) from about 0.01 to about 1.0 wt. % cationic tenside.

17. Cosmetic composition according to claim 16, wherein the cationic tenside is further defined as a quaternary ammonium compound.

18. Cosmetic composition according to claim 17, wherein the quaternary ammonium compound is further defined as a cetyltrimethyl ammonium chloride.

19. Cosmetic composition according to claim 8, wherein the composition comprises a propellant selected from the group of propane, propane/butane mixtures, dimethyl ether, or compositions thereof.

20. Method according to claim 10, wherein the method comprises:
applying the cosmetic composition to human hair for a period of from about 1 to about 10 minutes; and
removing the cosmetic composition from the hair.
